(19) United States
(12) Patent Application Publication
(10) Pub. No.: US 2005/0267213 A1
(43) Pub. Date: Dec. 1, 2005

(54) Use of photodynamic therapy to enhance treatment with immuno-modulating agents

(75) Inventors: Michael Gold, Nashville, TN (US); Mitchel Goldman, La Jolla, CA (US); Scott Lundahl, Lexington, MA (US); Paul Sowyrda, Medfield, MA (US)

Correspondence Address: FOLEY AND LARDNER;SUITE 500, 3000 K STREET NW, WASHINGTON, DC, 20007, US

(73) Assignee: DUSA PHARMACEUTICALS, INC.

(21) Appl. No.: 11/030,311

(22) Filed: Jan. 7, 2005

Related U.S. Application Data
(60) Provisional application No. 60/534,701, filed on Jan. 8, 2004.

(51) Int. Cl. A61K 31/198

(52) U.S. Cl. 514/561; 604/20

(57) ABSTRACT

Methods of enhancing the efficacy and safety of treatment with immuno-modulating agents are provided. The inventive methods combine treatment with immuno-modulating agents with photodynamic therapy (PDT). PDT can be administered before, during or after administration of the immuno-modulating agent. Combination therapy achieves at least the same efficacy as immuno-modulatory therapy in less time, with less immuno-modulating agent and fewer side-effects.

### FIELD OF INVENTION

The invention relates to the treatment of diseases using immuno-modulating agents and photodynamic therapy (PDT).

### BACKGROUND OF THE INVENTION

A group of compounds, collectively referred to as “immuno-modulating agents,” have been developed that can modify responses in the immune system. These compounds modulate the immune system by interfering with T-cell interactions or impeding the function of various cytokines. For example, alefacept (i.e. AMEVIVE®, Biogen, Inc., Cambridge, Mass.) is an immunosuppresive dimeric fusion protein that consists of the extracellular CD2-binding portion of the human leukocyte function antigen-3 (LFA-3) linked to the Fc (hinge, CH2 and CH3 domains) portion of human IgG1. Alefacept interferes with lymphocyte activation by specifically binding to the lymphocyte antigen CD2 and inhibiting LFA-3/CD2 interaction. Alefacept also causes a reduction in subsets of CD2+ T lymphocytes (primarily CD45RO+), presumably by bridging between CD2 on target lymphocytes and immunoglobulin Fc receptors on cytotoxic cells, such as natural killer cells. Treatment with alefacept results in a reduction in circulating total CD4+ and CD8+T lymphocyte counts. Alefacept is currently approved for treating psoriasis.

Another example of an immuno-modulating agent is infliximab (i.e. REMICADE®, Centocor, Malvern, Pa.). Infliximab is a chimeric monoclonal antibody against tumor necrosis factor-(alpha) (TNF-α). When fighting diseases such as rheumatoid arthritis and Crohn's disease, the immune system produces excess TNF-α, which can lead to inflammation and the degradation of healthy tissues. Infliximab specifically binds TNF-α and thereby reduces the inflammation associated with excess TNF-α and prevents permanent damage to the body's bones, cartilage and tissue. Infliximab is approved for treating rheumatoid arthritis and Crohn's disease.

Etanercept (i.e. ENBREL®, Immunex Corp., Thousand Oaks, Calif.) is yet another useful immuno-modulating agent. Etanercept is a dimeric fusion protein consisting of the extracellular ligand-binding portion of the human 75 kilodalton (p75) tumor necrosis factor receptor (TNFR) linked to the Fc portion of human IgG1. Entanercept binds specifically to TNF and blocks its interaction with cell surface TNF receptors. As entanercept is a dimeric soluble form of the p75 TNFR, it can bind two TNF molecules and inhibits binding of both TNFα and TNFβ to cell surface TNFRs, rendering TNF biologically inactive. Entanercept is approved for treating rheumatoid arthritis, juvenile rheumatoid arthritis, psoriatic arthritis and ankylosing spondylitis.

In addition, many imidazoquinoline amine, imidazopyridine amine, 6,7-fused cycloalkylimidazopyridine amine, 1,2-bridged imidazoquinoline amine, thiazolo- and oxazolo-quinolinamines and pyridinamines, imidazonaphthyridine and tetrahydroimidazonaphthyridine amine compounds have demonstrated potent immunostimulating, antiviral and antitumor (including anticancer) activity, and have also been shown to be useful as vaccine adjuvants to enhance protective immune system response to vaccines. Such compounds are disclosed in, for example, U.S. Pat. Nos. 4,689,338, 5,389,640, 5,268,376, 4,929,624, 5,266,575, 5,352,784, 5,494,916, 5,482,936, 5,346,905, 5,395,937, 5,238,944, 5,525,612, and WO99/29693 wherein their immunostimulating, antiviral and antitumor activities are discussed in detail, and certain specific diseases are identified as being susceptible to treatment therewith, including basal cell carcinoma, eczema, essential thrombocythaemia, hepatitis B, multiple sclerosis, neoplastic diseases, psoriasis, rheumatoid arthritis, type I herpes simplex, type II herpes simplex, and warts. One of these compounds, known as imiquimod, has been commercialized in a topical formulation, Aldara™, for the treatment of anogenital warts associated with human papillomavirus. The mechanism for these compounds is thought to be due in substantial part to enhancement of the immune response due to induction of various important cytokines (e.g., interferons, interleukins, tumor necrosis factor, etc.). Such compounds have been shown to stimulate a rapid release of certain monocyte/macrophage-derived cytokines and are also capable of stimulating B cells to secrete antibodies which play an important role in these compounds' antiviral and antitumor activities. One of the predominant immunostimulating responses to these compounds is the induction of interferon (IFN)-alpha. production, which is believed to be very important in the acute antiviral and antitumor activities seen. Moreover, up regulation of other cytokines such as, for example, tumor necrosis factor (TNF), IL-1 and IL-6 also have potentially beneficial activities and are believed to contribute to the antiviral and antitumor properties of these compounds.

While immuno-modulating agents offer great promise for treating diseases such as rheumatoid arthritis and psoriasis, the compounds are slow acting. Initial control of the disease state can require extensive treatment. Furthermore, the compounds possess serious side effects and are very expensive.

Accordingly, new methods of treating diseases that are less expensive and well-tolerated are required.

### SUMMARY OF THE INVENTION

It is, therefore, one object of the present invention to provide methods of treating autoimmune diseases.

It is also an object of the present invention to provide methods of treating conditions related to hyperproliferative cells.

It is another object to provide methods of treating malignant lesions.

It is also an object of the present invention to provide methods of enhancing the efficacy of an immuno-modulating agent.

In accomplishing these and other objects of the invention, there is provided, in accordance with one aspect of the invention, a method of treating autoimmune disease in a patient comprising administering to said patient an immuno-modulating agent and a photosensitizer and exposing said patient to light. The treatment also can be used to treat malignant lesions and conditions related to hyperproliferative cells. In a preferred embodiment, the photosensitizer is aminolevulinic acid (ALA). Similarly, preferred immuno-modulating agents include alefacept, infliximab, etanercept and imiquimod. Blue or red light is the preferred photoactivating light.

In another aspect, there is provided a method of enhancing the efficacy of an immuno-modulating agent comprising administering to a patient undergoing therapy with the immuno-modulating agent a therapeutically effective amount of a photosensitizer and exposing the patient to blue light. In a preferred embodiment, the photosensitizer is ALA.

Also provided is a method of enhancing the safety of an immuno-modulating agent comprising administering to a patient undergoing therapy with an immuno-modulating agent a photosensitizer and exposing the patient to light.

Other objects, features and advantages of the present invention will become apparent from the following detailed description. The detailed description and specific examples, while indicating preferred embodiments, are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### DETAILED DESCRIPTION

The effect of immuno-modulating agents can be enhanced by combining the treatment with photodynamic therapy (PDT). PDT can be administered before, during or after administration of the immuno-modulating agent. Combination therapy achieves at least the same efficacy as immuno-modulatory therapy in less time, with less immuno-modulating agent and fewer side-effects.

While the mechanism by which the invention operates is not fully understood, and the inventors do not wish to limit their invention to any particular theory, it is believed that PDT induces local and systemic immuno-modulating effects and that said activity can be combined with an immuno-modulating agent to rapidly control the immune system with less side effects than immuno-modulating agents alone.

### DEFINITIONS

The term “immuno-modulating agent” refers to compounds that can alter normal responses in the immune system. The alterations can result in either suppressed or stimulated immune responses.

The term “photosensitizer,” as used herein, refers to compounds that can themselves absorb light and lead to formation of singlet oxygen or that can induce the synthesis or accumulation or both of protoporphyrin IX (PpIX) and other endogenous porphyrins, their precursors and their photoproducts. Thus, the term encompasses photosensitizers per se, as well as photosensitizer precursors.

The term “autoimmune disease” denotes conditions wherein an individual's immune system mistakenly targets the cells, tissues, and organs of the individual's own body.

“Porphyrin(s)” and their precursors refer to compounds produced in vivo in the synthesis of heme and other endogenously produced photoactivatable compounds including their photoproducts.

By the expression “rapidly growing cell” is meant herein any lesion, abnormal cell or normal cell that exhibits cell growth substantially greater than that of the surrounding tissues and that preferentially accumulates protoporphyrin IX from exogenous ALA.

### DISCUSSION

In one embodiment, the invention provides a method of treating disease, such as an autoimmune disease, in a patient comprising administering to the patient an immuno-modulating agent and a photosensitizer and exposing the patient to light. In a preferred embodiment, the patient is administered the photosensitizer and exposed to the light before being administered the immuno-modulating agent. Alternatively, the administration of the photosensitizer and light can occur simultaneously with or after the immuno-modulatory treatment.

Photodynamic therapy (PDT) consists of the topical, oral or systemic application of a photosensitizer, which can absorb light in the visible spectrum and subsequent irradiation with light of the corresponding wavelength. Irradiation results in the formation of singlet oxygen, which is toxic to cells.

Porphyrin is one example of a photosensitizer. Porphyrins and related photosensitizers, are given systemically (by intravenous injection), and they also are given either topically or by intralesional injection. They can be activated by visible light at wavelengths that are absorbed by the porphyrins molecule. Historically, red light has been used, but activation is possible by any such absorbed wavelength. They can be activated by visible (red) light. The localized exposure of porphyrin-containing tissues to such light ordinarily does not induce a chemical reaction between cell components and the porphyrin molecules. Instead, the porphyrins act as catalysts by trapping the energy of the photoactivating light and then passing it on to molecules of oxygen, which in turn are raised to an excited state that is capable of oxidizing adjacent molecules or structures. Cell death is not caused primarily by damage to the DNA, but by damage to essential membrane structures. The goal of photochemotherapy is sometimes cure (mainly for basal cell carcinomas), but usually the goal is palliation through local control when none of the standard forms of therapy are considered likely to offer a significant degree of benefit to the patient.

At present, the porphyrins most commonly used for photochemotherapy are Hematoporphyrin IX (HpIX), Hematoporphyrin derivative (HpD) and various semi-purified preparations of HpD such as commercially available Photofrin® II, a semi-purified form of HpD. When porphyrins are used as photosensitizers, cell death results from damage to cell membranes. Consequently, malignant transformation is not a serious problem. Moreover, since the visible (red) light that is used to photoactivate porphyrins penetrates tissue much more deeply than does the ultraviolet light that must be used to photoactivate methoxypsoralens, the depth at which porphyrin-treated tissue can be killed is substantially greater. Also, since certain types of porphyrins show a significant tendency to accumulate preferentially in malignant tissues, it is sometimes possible to destroy malignant tissue without causing clinically significant damage to adjacent normal tissues.

The main problem with the systemic use of HpIX, HpD and Photofrin II is that photosensitizing concentrations persist in the skin for several weeks to several months following their administration. Consequently, severe accidental phototoxic skin reactions may occur unless the patient avoids exposure to sunlight (either direct, or filtered through window glass) until the concentration of the photosensitizer in the skin has been reduced to a harmless level. At present, the problem of photosensitivity following the administration of porphyrins is handled by advising the patient to avoid any form of exposure to sunlight (or to very bright artificial lights) for a period of at least two weeks post-injection, and to initiate subsequent exposure to sunlight very cautiously. Not all patients comply with these instructions, since it often is quite inconvenient to do so. In addition, the use of a sunscreen with a high blocking factor is recommended with warning that this will only reduce the hazard somewhat, not eliminate it completely. In a few cases, patients whose photosensitization persisted for more than a month post-treatment have been given large daily doses of beta-carotene over a period of several months in an attempt to prevent accidental phototoxic damage. Finally, attempts have been made to reduce phototoxicity by applying the photosensitizer topically to a limited area.

However, another type of problem is encountered if HpIX or HpD is applied topically in DMSO (dimethylsulfoxide), Azone, or some other vehicle intended to enhance their diffuision through tissue. The porphyrins tend to become immobilized wherever they happen to be when the DMSO or Azone becomes diluted by normal tissue fluids to such an extent that the porphyrins can no longer diffuse through the tissue (or even remain in solution). Consequently, the topical application of porphyrins often is associated with a loss of specificity for malignant tissues, and normal tissues near the site of application may develop persistent photosensitization from the localized concentration of porphyrin.

Alternatively, a photodynamic (photosynthesizing) treatment method can be employed which uses an agent which can be administered either systemically or topically which is not in itself a photosensitizer but which induces the synthesis or accumulation or both of protoporphyrin IX (PpIX) and other endogenous porphyrins, their precursors and their photoproducts, in rapidly growing cells, including abnormal cells in otherwise normal tissues, in vivo or in vitro.

Protoporphyrin IX (PpIX), a naturally occurring photosensitizer, is the immediate precursor of heme in the heme biosynthetic pathway. All nucleated cells have at least a minimal capacity to synthesize PpIX, since heme is necessary for the synthesis of various essential heme-containing enzymes. Certain types of cells and tissues can synthesize relatively large quantities of PpIX. Under normal conditions, the synthesis of PpIX in such tissues is under such tight feed-back control that the cells produce it at a rate just sufficient to match their need for heme. However, the usual rate-limiting step in the process, the synthesis of 5-Amino-4-oxopentanoic acid, also known as 5-aminolevulinic acid, (“ALA”), can be bypassed by the provision of exogenous ALA, porphobilinogen, or other precursor of PpIX. Certain tissues and organs will then accumulate such a large excess of PpIX that they become both fluorescent and photosensitive. At least in the case of the skin, the PpIX appears to be synthesized in situ. ALA has been used in conjunction with PDT to detect and treat rapidly growing cells. See e.g. U.S. Pat. Nos. 5,422,093; 5,211,938; 5,079,262, which are hereby incorporated by reference.

Thus, in a preferred embodiment, the PDT portion of the inventive therapies comprises administration of the photosensitizer precursor ALA and the use of light. The ALA can be administered parenterally, orally or topically. ALA is commercially available from Sigma Chemical Company and other sources and which is water soluble. The oral and parenteral routes lead to the induction of clinically useful concentrations of PpIX in certain benign and malignant tissues throughout the body.

A “therapeutically effective” amount of the immuno-modulating agent and photosensitizer can be determined by prevention or amelioration of adverse conditions or symptoms of the diseases, injuries or disorders being treated. In one example, the amounts of ALA constituting an effective dose can be determined by one skilled in the art by analogy with the doses used for synthetic porphyrins, based on milligrams per kilogram body weight for in vivo systemic application and the typical concentrations for topical or ex vivo applications. The compound can be conveniently used orally or intravenously at a dosage of about 10 to 100 mg/kg per single dose, preferably as a dosage of 40-50 mg/kg; however split dosages of 10 mg/kg four times per day may also be given. The compound can be used topically at a dose of between 2% to 100%, with 100% being dry powder. Ex vivo concentrations of the compound are used on cell suspensions in a range of 1-5 mM, with a preferred range of 1-2 mM; however, if serum is present, a higher dose of about 15 mM should be used. If ex vivo use on whole blood, the compound is used at about 15 mM; however, if an iron kelator, such as Desferol™ or des ferroxamine, a lower concentration may be used.

In one aspect of the invention, blue or red light is used in the PDT portion of the inventive therapy. A preferred wavelength of the photoactivating light is in the range of 350 to 700 nm. In other embodiments the photoactivating light is in the range of 375 to 450 nm, 600 to 700 nm, 625 to 670 nm, or 625 to 640 nm.

In a preferred embodiment, the patient is administered the photosensitizer and exposed to the light before being administered the immuno-modulating agent. The preliminary administration of PDT effectively primes the immune system, enabling efficacious results to be achieved more rapidly using less immuno-modulating agent, thereby reducing costs and potential side effects.

Alternatively, the patient can be administered the photosensitzer before or with the immuno-modulating agent and be irradiated during the course of the therapy with the immuno-modulating agent. Such a dosing regimen achieves similar or greater clinical efficacy with lower doses of immuno-modulating agent.

In another embodiment, the patient receives the PDT after disease control has been achieved with the immuno-modulating agent. This strategy permits a dose reduction of the immuno-modulating agent while maintaining the clinical efficacy.

The inventive methods can be used to treat a variety of diseases. In one aspect of the invention, the inventive methods can be used to treat autoimmune diseases. Examples of treatable autoimmune diseases include, but are not limited to, psoriasis, rheumatoid arthritis, psoriatic arthritis, ankylosing spondylitis, Crohn's disease, systemic lupus erythematosus, scleroderma and rosacea, Felty's syndrome, CREST syndrome, dermatomyositis, thyroiditis, and Sjgren's syndrome. The inventive methods also can be used to treat malignant lesions, warts and conditions related to hyperproliferative cells. In a preferred embodiment, the inventive methods are used to treat skin diseases. For example, the inventive methods can be used to treat malignant and non-malignant tissue abnormalities and lesions of the skin; conjunctiva; respiratory, digestive and vaginal mucosa; endometrium; and urothelium.

Without further elaboration, it is believed that one skilled in the art can, using the preceding description, utilize the present invention to its fullest extent. In carrying out the method of this invention, the quantities of materials utilized are not in themselves critical and can be varied within the scope and spirit of the invention. The following examples are merely illustrative of preferred embodiments and not intended to be limitative of the remainder of the disclosure in any way whatsoever.

### EXAMPLES

1. Use of Combination Therapy to Treat Psoriasis

Several patients presenting symptoms of psoriasis were topically administered ALA and subsequently exposed to light from an Intense Pulsed Light (IPL) device. Patients were then treated with alefacept as would normally be done. Whereas alefacept therapy normally requires approximately eight weeks to improve the treated condition, the combination therapy produced almost immediate improvements.

2. Use of Combination Therapy to Treat Psoriasis

A patient who has just completed a treatment cycle with alefacept for psoriasis is topically administered ALA and subsequently exposed to blue light. The patient experiences less return of their psoriatic symptoms due to the cessation of alefacept therapy.

3. Use of Combination Therapy to Treat Basal Cell Carcinoma

A patient with a Basal Cell Carcinoma is administered ALA in the form of a topical cream, which is applied to the affected areas. Several hours after administration of the ALA, the patient is exposed to an activating red light. Subsequently, the patient is administered a course of imiquimod therapy. The patient experiences rapid and complete resolution of the cancerous lesion.

4. Use of Combination Therapy to Treat Psoriasis

A patient with psoriasis is administered ALA in the form of a topical cream, which is applied to the affected areas. Several hours after administration of the ALA, the patient is exposed to an activating light. Subsequently, the patient is administered a reduced course of alefacept therapy. The patient begins to experience an alleviation of symptoms soon after receiving the treatment.

5. Use of Combination Therapy to Treat Psoriasis

A patient with psoriasis is administered ALA via i.v. injection. One to three hours after administration of the ALA, the patient is exposed to 5 J/cm² of 417 nm blue light. Subsequently, the patient is administered a reduced course of alefacept therapy. The patient begins to experience an alleviation of symptoms soon after receiving the treatment.

6. Use of Combination Therapy to Treat Psoriasis

A patient with psoriasis is orally administered ALA. One to three hours after administration of the ALA, the patient is exposed to 10 J/cm² of 417 nm blue light. Subsequently, the patient is administered a reduced course of alefacept therapy. The patient begins to experience an alleviation of symptoms soon after receiving the treatment.

7. Use of Combination Therapy to Treat Warts

A patient with warts is administered ALA in the form of a topical cream, which is applied to the affected area. Several hours after administration of the ALA, the patient is exposed to white light. Subsequently, the patient is administered a reduced course of imiquimod therapy. The patient begins to experience an alleviation of symptoms soon after receiving the treatment.

1. A method of treating autoimmune disease in a patient comprising administering to said patient an immuno-modulating agent and a photosensitizer and
exposing said patient to light.

2. The method of claim 1, wherein said disease is psoriasis.

3. The method of claim 1, wherein said immuno-modulating agent is an immuno-suppressive agent.

4. The method of claim 1, wherein said immuno-modulating agent is a protein based immuno-suppressive agent.

5. The method of claim 1, wherein said immuno-modulating agent is an immuno-enhancing agent.

6. The method of claim 1, wherein said immuno-modulating agent is a protein based immuno-enhancing agent.

7. The method of claim 1, wherein said photosensitizer is aminolevulinic acid (ALA).

8. The method of claim 1, wherein said photosensitizer is a chemical salt or other chemical derivative of aminolevulinic acid.

9. The method of claim 1, wherein said photosensitizer is applied topically.

10. The method of claim 1, wherein administration of said photosensitizer and exposure of said patient to light occurs prior to administration of said immuno-modulating agent.

11. The method of claim 1, wherein administration of said photosensitizer and exposure of patient to light occurs after administration of said immuno-modulating agent.

12. The method of claim 1, wherein administration of said photosensitizer and exposure of said patient to light is concurrent with administration of said immuno-modulating agent.

13. The method of claim 1 wherein the light is blue light.

14. The method of claim 1 wherein the light is red light.

15. The method of claim 1 wherein the light is pulsed light.

16. The method of claim 1 wherein the light is laser light.

17. A method of treating a malignant lesion in a patient comprising administering to said patient an immuno-modulating agent and a photosensitizer and
exposing said patient to light.

18. A method of treating a disease in a patient comprising administering to said patient an immuno-modulating agent and a photosensitizer and
exposing said patient to light,
wherein said disease is a condition related to hyper-proliferative cells.

19. A method of enhancing the efficacy of an immuno-modulating agent comprising administering to a patient undergoing therapy with an immuno-modulating agent a photosensitizer and
exposing said patient to light.

20. The method of claim 19, wherein said therapy is directed at treating psoriasis.

21. The method of claim 19, wherein said therapy is directed at treating a malignant lesion.

22. The method of claim 19, wherein said therapy is directed at treating a condition related to hyper-proliferative cells.

23. The method of claim 19, wherein said immuno-modulating agent is an immuno-suppressive agent.

24. The method of claim 19, wherein said immuno-modulating agent is a protein based immuno-suppressive agent.

25. The method of claim 19, wherein said immuno-modulating agent is an immuno-enhancing agent.

26. The method of claim 19, wherein said immuno-modulating agent is a protein based immuno-enhancing agent.

27. The method of claim 19, wherein said photosensitizer is aminolevulinic acid (ALA).

28. The method of claim 19, wherein said photosensitizer is a chemical salt or other chemical derivative of aminolevulinic acid.

29. The method of claim 19, wherein said photosensitizer is applied topically.

30. The method of claim 19, wherein administration of said photosensitizer and exposure of said patient to light occurs prior to administration of said immuno-modulating agent.

31. The method of claim 19, wherein administration of said photosensitizer and exposure of patient to light occurs after administration of said immuno-modulating agent.

32. The method of claim 19, wherein administration of said photosensitizer and exposure of said patient to light is concurrent with administration of said immuno-modulating agent.

33. The method of claim 19, wherein the light is blue light.

34. The method of claim 19, wherein the light is red light.

35. The method of claim 19, wherein the light is pulsed light.

36. The method of claim 19, wherein the light is laser light.
